(19) United States
(12) Patent Application Publication
(10) Pub. No.: US 2026/0030745 A1
(43) Pub. Date: Jan. 29, 2026

(54) Oncological Foundation Models, Systems, and Methods

(71) Applicant: Picture Health, Inc., Cleveland, OH (US)

(72) Inventors: Nathaniel BRAMAN, Cleveland, OH (US); Amogh HIREMATH, Pittsburgh, PA (US)

(21) Appl. No.: 18/786,417

(22) Filed: Jul. 26, 2024

(51) Int. Cl. G06T 7/00; G06V 10/44; G16H 50/20; G16H 50/70

(52) U.S. Cl. CPC G06T 7/0012; G06V 10/44; G16H 50/20; G16H 50/70; G06T 2207/30024; G06T 2207/30096

(57) ABSTRACT

An oncological foundation model is trained with broad, multimodal data to make predictions concerning a variety of different types of cancers. For example, the foundation model may make use of medical images drawn from radiology and pathology, as well as immunohistochemistry data; the presence or absence of biomarkers for particular diagnoses; patient history data; patient demographic data; and other forms of medical data. When using medical images, whole medical images as well as feature sets derived from the medical images may be used. The foundation model may have both causal predictive abilities as well as generative abilities.

### TECHNICAL FIELD

The invention relates generally to the field of artificial intelligence, and more specifically to foundation models for the diagnosis and treatment of cancers.

### BACKGROUND

“Artificial intelligence” (AI) is a broad term generally referring to the use of trained machine-learning models to make predictions, recommendations, and decisions in a variety of real-world situations. AI has innumerable applications, ranging from the prosaic to the specialized, some of the most promising of which lie in medicine. Medical AI systems may be able to see patient data in ways that human medical providers cannot, make connections that human medical providers cannot, and improve the treatment of patients.

There have been some notable successes in medical AI. For example, in the field of radiomics, quantitative data is extracted from medical images and is used by machine learning models to assist in diagnoses, prognoses, and treatment decisions. Such systems have shown promise, for example, in determining whether a particular patient is likely to respond to immunotherapy treatments, and whether a particular patient is likely to experience certain side effects of treatment. Useful clinical predictions can also be made by machine learning models that use pathology images, such as whole slide images (WSIs) of tissue samples, a field often called pathomics. There are also techniques that fuse radiomics and pathomics to make predictions. U.S. Pat. Nos. 9,483,822 and 9,767,555 provide relatively early examples of radiomic and radiomic-pathomic methods for making medical predictions using machine learning models.

Despite these successes, the machine learning models used in the patents described above are relatively limited in scope and capabilities. These machine learning models, and machine learning models like them, operate using specific, limited types of data, and are consequently able to provide only very limited types of predictions based on that data, e.g., whether a particular patient's lung tumor will respond to a specific drug or type of drug, like an immune checkpoint inhibitor. In these applications, the machine learning model itself is often a simple classifier.

Although so-called “deep learning” machine models (i.e., machine models that use artificial neural networks, such as convolutional neural networks (CNNs)) are used in medical AI, these models have some downsides. For example, they require a great deal of data to train, and results from these models may not be readily interpretable, i.e., it may be difficult for a clinician to discern why the deep learning machine model is making a particular prediction or offering a particular result.

### BRIEF SUMMARY

One aspect of the invention relates to an oncological foundation model. The oncological foundation model is trained to make predictions regarding two or more different types of cancer using two or more different types or modalities of input data.

Most often, the foundation model will be a deep learning model trained to make predictions on ten or more types of cancer using broad, multimodal data, e.g., combinations of available data, including radiology data and images; pathology data and images; genetic data; immunohistochemistry data; the presence or absence of biomarkers for particular diagnoses; patient history data; patient demographic data; and other forms of medical data.

The architecture of the foundation model may be chosen to maximize flexibility with disparate types of data. For example, the foundation model may have a transformer-based architecture in at least some embodiments. In general, the foundation model is designed and trained to maximize transparency and interpretability. For example, in general, the foundation model is built with as few layers as possible, and as few parameters as needed for the range of predictions it is expected to make. To minimize the need for training data, the foundation model may be trained using a combination of supervised and self-supervised learning.

In making predictions, the foundation model may use any combination of whole medical images and features extracted from those medical images. When using extracted image features, the foundation model may be trained to use feature sets that have an established association with particular biological phenomena or effects.

The foundation model may make use of causal prediction, e.g., to compare the effects of several potential treatments. Using causal prediction, the foundation model may suggest a particular course of treatment.

Other aspects, features, and advantages of the invention will be set forth in the following description.

### BRIEF DESCRIPTION OF THE DRAWING FIGURES

The invention will be described with respect to the following drawing figures, in which like numerals represent like features throughout the description, and in which:

FIG. 1 is a schematic diagram of a system including an oncological foundation model and its related components;

FIG. 2 is a detailed schematic of a preprocessor/featurizer element of FIG. 1;

FIG. 3 is an illustration of a graphical user interface used to select input data for the foundation model of FIG. 1;

FIG. 4 is an illustration of another graphical user interface used to provide input to the foundation model of FIG. 1;

FIG. 5 is an illustration of an input preprocessing element that takes natural language input;

FIG. 6 is a schematic flow diagram of a method for training the foundation model of FIG. 1; and

FIG. 7 is a schematic flow diagram of a method for making and updating longitudinal predictions.

### DETAILED DESCRIPTION

### Concepts and Definitions

This description is directed to the characteristics, training, and implementation of medical foundation models and related models and systems that can be used to make a wide variety of medical predictions given a variety of different types of medical data. In this description, the terms “model,” “machine model,” and “machine learning model” are used interchangeably to refer to a computer program that has been trained to make a prediction or predictions based on various types of input data. Machine learning models may be of various types, and unless the type is specified, the term should be considered to be generic. For example, a “deep learning model” is a machine model that uses an artificial neural network, such as a convolutional neural network (CNN), to make its predictions.

While the definition of “foundation model” varies somewhat depending on the authority one consults, for purposes of this description, the term refers to a machine model that is trained on broad data, generally using unsupervised or self-supervised learning, and is applicable across a wide range of contexts. Some authorities describe a foundation model by the number of parameters the model uses, i.e., the number of individually-weightable variables that define the model's output given a set of inputs. For example, some authorities require that a machine model have, e.g., tens of billions of parameters, to be considered a foundation model. (Sec, e.g., Safe, Secure, and Trustworthy Development and Use of Artificial Intelligence, Executive Order 14110 of Oct. 30, 2023, 88 Fed. Reg. 75191, 75194 (Nov. 1, 2023).) However, a medical foundation model in this description may have fewer parameters, e.g., on the order of hundreds of millions to billions of parameters, depending on the intended scope of its capabilities. Moreover, while unsupervised and self-supervised learning techniques may be used to train medical foundation models according to this description, as will be described below, the medical foundation models described here may use at least some supervised learning in some embodiments.

Although the techniques described here can be used to create many different types of medical foundation models, the remainder of this description assumes that the foundation models are oncological foundation models. In that context, “broad data” refers to two or more different types of data, such as radiology data and images; pathology data and images; genetic data; immunohistochemistry data; the presence or absence of biomarkers for particular diagnoses; patient history data; patient demographic data; and other forms of medical data. Foundation models according to this description may also be trained on or use longitudinal data of various types and origins, by which this description means data taken on the same patient or group of patients over time, such as radiology and pathology images taken at various points during the course of patient monitoring, diagnosis, and treatment.

The foundation models described here may be applicable to a broad range of different types of cancers (e.g., lung cancer, breast cancer, ovarian cancer, colon cancer, oropharyngeal cancer, pancreatic cancer, etc.), and may be able to provide useful predictive output in, e.g., diagnosis, defining and ranking treatment options, risk stratification, predicting response to particular drugs or treatments, formulating treatment plans, etc. In each case, the foundation models described here may provide an indication of confidence in any predictions that are made and may be trained to provide a prediction of what additional data or forms of data are likely to improve the confidence level of a prediction.

The term “prediction” is used in this description to describe most forms of output from the kinds of medical foundation models described here. This is because the output of all machine models is, at some level, uncertain and predictive in nature. That is, the machine model is essentially guessing the correct output to any input based on similar training data. A “longitudinal prediction” is a prediction that concerns the evolution or progression of a patient or group of patients over time. A longitudinal prediction may or may not use longitudinal data. For example, a longitudinal prediction may use patient data from one or several points in time to predict the evolution of a patient's disease over time.

In this description, the term “medical image” is a generic term that refers to radiology images, pathology images and other forms of imagery, like documentary photographs and micrographs, taken for either clinical or research purposes. A medical image may be individual in nature, or it may be a slice, projection, or other part of a larger medical imaging study. Medical images include, e.g., images derived from X-ray studies, ultrasound studies, CT scans, MRI scans, and PET scans, as well as images derived from specialized applications of those kinds of imaging modalities, like mammography and breast tomosynthesis. Medical images also include, e.g., whole-slide images (WSIs) of tissue samples acquired and examined in pathology studies and images derived from tissue microarrays. In many applications, the medical images will be routine clinical images, acquired in the normal course of diagnosing, treating, and monitoring patients. In acquiring these routine medical images, typically, the priority is on routine clinical practice-producing something that a radiologist, pathologist, or other medical professional can review to provide useful clinical or research information, rather than acquiring specialized images specifically for use with a machine model. Thus, a foundation model as described here is preferably trained to work with whatever medical images are available.

To give a particular example of the use of routine clinical imaging, in pathology, perhaps the most common tissue stain used in preparing pathology specimens for examination is hematoxylin and eosin (H&E) stain, and for that reason, a foundation model that is trained to accept pathology images would usually be trained with H&E-stained tissue images, such as whole-slide images (WSIs) of H&E-stained tissue. However, this is not to say that the training of a foundation model should be limited to the most common types of input, or that all medical images used with a foundation model need be routine clinical images.

The term “lesion,” as used here, refers to any kind of damage to, or disease in, tissue. A lesion may be benign, or it may be malignant (i.e., cancerous). Thus, the term “lesion” should be thought of as generic—for example, a cancerous solid tumor is a malignant form of lesion.

### Foundation Model Embodiments, Applications, and Systems

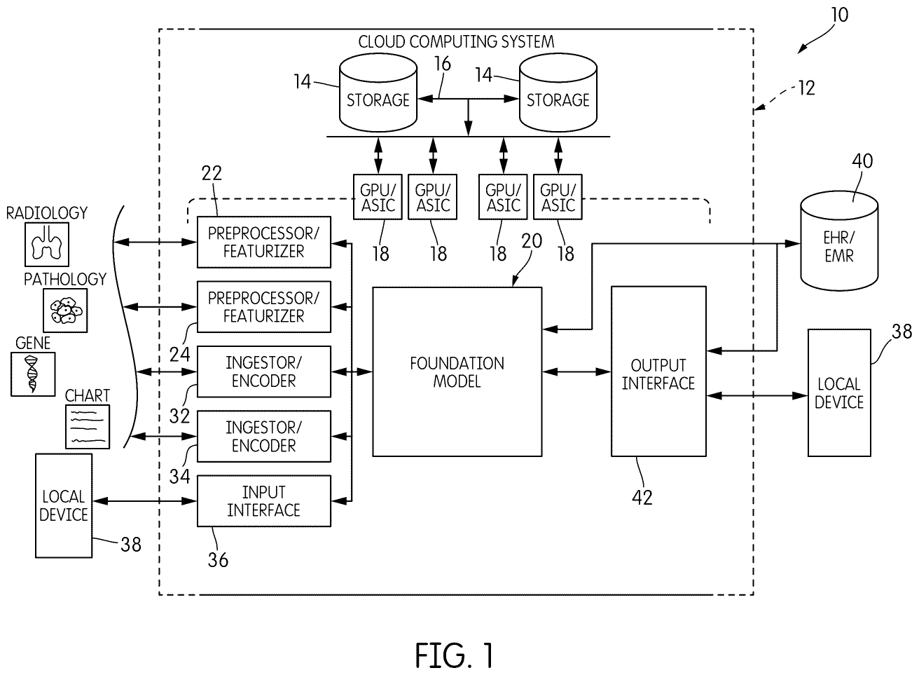

FIG. 1 is a schematic diagram of a system, generally indicated at 10, according to one embodiment of the invention. System 10 is typically implemented using a cloud computing system 12, i.e., a large-scale system physically implemented in a data center or other dedicated facility that is used through a computer network, such as the Internet. The cloud computing system 12 generally includes one or more storage devices 14, a data bus 16 or other hardware for interconnecting components, and one or more processors 18. A typical cloud computing system 12 includes numerous processors 18, numerous storage devices 14, etc., all connected together and in communication with one another. The processors 18 may be general-use microprocessors, but in many embodiments, the processors 18 will be computer processing elements that are more capable or more specialized for AI use, like graphics processing units (GPUs), or application-specific integrated circuits (ASICs), like tensor processing units (TPUs). The cloud computing system 12 would typically also be equipped with memory, such as random-access memory (RAM) and read-only memory (ROM), although for simplicity, these are not shown in FIG. 1.

The other components shown within the cloud computing system 12 in FIG. 1 are software modules. That is, they are comprised of machine-readable instructions (i.e., software code) that, when executed by machines like the processors 18 and their connected components, cause the processors 18 to perform the functions described here. The machine-readable instructions are typically stored on the storage devices 14, although while the processors 18 are executing the instructions, instructions may be stored in a temporary form of memory, like RAM.

Although a cloud computing system 12 is described here and shown in FIG. 1, that does not necessarily preclude system 10 from being installed in a particular location for use at that location (i.e., an on-site installation). System 10 could, in at least some embodiments, be installed at a single location (e.g., a hospital) for use at that location. However, as those of the art will understand, the amount of computing hardware necessary, the amount of space required, and other considerations, like power consumption, generally make it more convenient for systems like system 10 to be cloud-based.

At the heart of system 10 is the foundation model 20 itself. The foundation model 20 is a deep-learning machine model. While the type of deep-learning machine model may vary somewhat from embodiment to embodiment, it is preferable if the foundation model 20 has a basic architecture that is flexible for use with heterogeneous data. For example, the foundation model 20 may be trained with radiology, pathology, genetic, and clinical data (i.e., “multimodal data”) to make predictions for a particular type of cancer. However, when the foundation model 20 is used, it preferably is able to base a prediction on any one or two of those types of data, using whatever data is available to it without requiring the input to include all data types on which the foundation model 20 was trained. The remainder of this description will assume that the foundation model 20 has a transformer-based architecture. Briefly, a typical transformer model combines a self-attention mechanism with a simple feed-forward artificial neural network to create an output.

The foundation model 20 may have any number of layers, and any number of hidden layers, in its architecture. As was alluded to above, the number of parameters used in the foundation model 20 is not particularly limited and may be of any order of magnitude, depending on the desired capabilities of the foundation model 20, although a number of parameters in the range of hundreds-of-millions to billions may be appropriate in at least some embodiments.

In designing and implementing the foundation model 20, certain things may be helpful to consider. First, because the foundation model 20 is medical in its applications, its use involves risk-primarily risk that a prediction or prediction-based recommendation made by the foundation model 20 is incorrect or incomplete, which could result in harm to a patient or a number of patients. For this reason, the foundation model 20 is preferably designed and implemented to maximize interpretability, i.e., the ability of the foundation model 20 to convey predictions and recommendations in such a way that clinicians and others can understand the basis or bases for those predictions and recommendations. In the illustrated embodiment, the desire to maximize interpretability influences the design of the foundation model 20, the way that the foundation model 20 and its associated components are implemented, and the way that predictions and recommendations are presented in actual use.

For example, it may be helpful to design the architecture of the foundation model 20 to be as small as possible, and to constrain the number of layers, and thus, the number of calculations that are applied between input and output. The attention mechanisms should be configured so that, to the maximum extent possible, the attention maps can be understood. The foundation model 20 may also be designed to calculate a measure of uncertainty, such as a confidence interval, with every prediction or recommendation that it makes. That measure of uncertainty may be presented in various ways to the end user, as will be described below in more detail.

The types of input used in training and during actual predictive use may also be selected to provide the most understandable, interpretable results, particularly in the training and use of medical images. Many prior deep learning models that process medical images use the whole image, and it may not always be clear which features, elements, or portions of the image are influencing the ultimate prediction or recommendation. Moreover, models that use whole images generally require more data to train properly, making training more of a burden.

By contrast, foundation models 20 may use an entire medical image or essentially unmodified medical data of some type as input, they may use specific features of the source medical image or data as input, or they may use both specific features and whole medical images or other unmodified data. Any combination of these things may be used to make any particular prediction or recommendation.

One advantage of using specific features of a medical image or other dataset as input is that those features can be designed and chosen according to an established association with a particular biological phenomenon or effect. For example, radiomic studies have demonstrated that measures and statistics descriptive of blood vessel curvature and tortuosity in the peri-tumoral region (i.e., the region around a tumor) are predictive of treatment response across a number of different cancers. (See, e.g., Braman, N., et al., “Novel Radiomic Measurements of Tumor-Associated Vasculature Morphology on Clinical Imaging as a Biomarker of Treatment Response in Multiple Cancers.” Clin. Cancer Res. 28 (20), pp. 4410-4424, (October 2022).) Often, there are biological hypotheses associated with these findings—for example, in the case of vessel curvature and tortuosity, it is hypothesized that if the tumor vasculature is more twisted and disorganized, it may prevent drug treatments from reaching the tumor. Thus, the use of features like this tends to ground any resulting prediction in established physiological relationships. Additionally, as will be described below in more detail, the use of known features, signatures, and biomarkers may reduce the amount of data needed to train the foundation model 20. Training the foundation model 20 to process features may also be a situation in which supervised learning is used.

When features of an image or other type of data are used, those features may be extracted by system 10, or they may be extracted by some other system at some other time. This description generally assumes that system 10 is responsible for extracting features from medical images and other data when those features are to be used by the foundation model 20. For that reason, the foundation model 20 of FIG. 1 is associated with a number of components whose purpose is to parse raw input data to extract features, statistics, and other descriptors of medical images and other medical data that serves as input to the foundation model 20. FIG. 1 shows two preprocessor/featurizer elements 22, 24, each one specialized for a particular type of preprocessing and feature extraction, although the foundation model 20 may make use of any number of preprocessor/featurizer elements 22, 24, each one adapted for a different type of feature, biomarker, or signature extraction from some kind of raw input data, like a medical image.

This description uses the term “feature” in this context to mean quantitative data extracted from medical images. When feature extraction is performed, features are often extracted in vast numbers, such that the extraction and processing of the features can only be performed by a machine, and the features themselves may be sub-visual, such that they can only be perceived by a machine.

Examples of radiomic features that may be used include histogram features, textural features, filter- and transform-based features, and size- and shape-based features, including the kinds of vessel features described above, which can be considered to be a special case of size- and shape-based features. The classification of various radiomic features may vary depending on the authority one consults; the categories used here should not be considered a limitation on the range of features that could potentially be used. The term “feature” should also be construed to include statistics that describe or summarize extracted features. For example, it may be convenient to use the mean, maximum, minimum, variance, skewness, kurtosis, etc. of a particular feature, globally or in a particular neighborhood, as input to a model. “Raw” features extracted from a medical image may also be normalized or otherwise manipulated before further use.

Histogram features use the global or local gray-level histogram, and include gray-level mean, maximum, minimum, variance, skewness, kurtosis, etc. Measures of energy and entropy may also be taken as histogram or first-order statistical features. Texture features explore the relationship between voxels and include the gray-level cooccurrence matrix (GLCM), the gray-level run-length matrix (GLRLM), gray-level size zone matrix (GLSZM), and gray-level distance zone matrix (GLDZM). Co-occurrence of local anisotropic gradient orientations (COLLAGE) features are another form of texture feature that may be used. (See Prasanna, P. et al., “Co-occurrence of local anisotropic gradient orientations (collage): distinguishing tumor confounders and molecular subtypes on MRI,” in Medical Image Computing and Computer-Assisted Intervention—MICCAI 2014 (eds. Golland, P. et al.), pp. 73-80 (Springer, 2014).) Filter- and transform-based features include Gabor features, a form of wavelet transform, and Laws features.

In addition to the kinds of vessel features described above, transform-based approaches to characterizing vessel features like curvature and tortuosity may also be used, such as Vascular Network Organization via Hough Transform (VaNgOGH). (Sec, e.g., Braman, N. et al., “Vascular Network Organization via Hough Transform (VaNgOGH): A Novel Radiomic Biomarker for Diagnosis and Treatment Response” in Medical Image Computing and Computer Assisted Intervention—MICCAI 2018 (eds. Frangi, A. F., et al.), pp. 803-811 (Springer, 2018)). As noted above, vessel features can be considered to be a special case of size- and shape-based features, considering the size and shape of the vessels, rather than the size and shape of the lesion.

Pathomic features may include, e.g., features of global and local graphs of the locations of nuclei, nuclear shape features, nuclear orientation entropy, and nuclear texture. Pathomic features may also include measures of other types of cells and structures, including, e.g., graphs and measures of tumor-infiltrating lymphocytes or measures of collagen fiber orientation, as well as statistics and graphs descriptive of these. Pathomic features may also include nuclear shape features, such as nuclear perimeter, minimum and maximum radii, smoothness, and Fourier transform of the nuclear contour (see, e.g., Lu, C. et al. “Nuclear shape and orientation features from H&E images predict survival in early-stage estrogen receptor-positive breast cancers,” Laboratory Investigation 98, pp. 1438-1448 June, 2018); nuclear texture features, such as gray-level co-occurrence features (Ibid.); global graphs of nuclei (see, e.g., Wang, X. et al. “Prediction of recurrence in early stage non-small cell lung cancer using computer extracted nuclear features from digital H&E images,” Scientific Reports 7:13543, October 2017); cell cluster graphs (i.e., local graphs, see, e.g., Ali, S. et al., “Cell cluster graph for prediction of biochemical recurrence in prostate cancer patients from tissue microarrays,” Proc. SPIE 8676, Medical Imaging 2013 March, 2013); cell orientation entropy (CORE; see, e.g., Lee, G. et al., “Cell Orientation Entropy (COrE): Predicting Biochemical Recurrence from Prostate Cancer Tissue Microarrays,” in Medical Image Computing and Computer Assisted Intervention—MICCAI 2013 (eds. Mori, K., et al.), pp. 396-403 (Springer, 2013)); local co-occurrence of cell morphology (LOCOM; see, e.g., Lu, C. et al., “A prognostic model for overall survival of patients with early-stage non-small cell lung cancer: a multicentre, retrospective study,” Lancet Digit. Health 2, e594-606, November 2020); feature-driven local cell clusters (FLocK; see Lu, C. et al., “Feature-driven local cell graph (FLocK): New computational pathology-based descriptors for prognosis of lung cancer and HPV status of oropharyngeal cancers,” Med. Image Analysis 68, November 2020); peri-nuclear pathomics (PNP; see, e.g., Wang, X. et al., “A prognostic and predictive computational pathology image signature for added benefit of adjuvant chemotherapy in early stage non-small-cell lung cancer,” eBioMedicine 69, July 2021); cell run length, which quantifies connectivity and branching patterns of cellular graphs; multinucleation index (MuNI; see, e.g., Koyuncu, C. et al., “Computerized tumor multinucleation index (MuNI) is prognostic in p16+ oropharyngeal carcinoma,” J. Clinical Investigation 131(8), March 2021); spatial interplay of tumor-infiltrating lymphocytes (SpaTIL; see, e.g., Corredor, G. et al., “Spatial Architecture and Arrangement of Tumor-Infiltrating Lymphocytes for Predicting Likelihood of Recurrence in Early Stage Non-Small Cell Lung Cancer,” Clin. Cancer Res. 25(5), March 2019); variations on SpaTIL for gynecologic cancers (ARCTIL; see, e.g., Azarianpour, S. et al., “Computational image features of immune architecture is associated with clinical benefit and survival in gynecological cancers across treatment modalities,” J. Immunother. Cancer 10(2), February 2022); variations on SpaTIL for oropharyngeal cancers (OP-TIL; see, e.g., Corredor, G. et al., “An imaging biomarker of tumor-infiltrating lymphocytes to risk-stratify patients with HPV-associated oropharyngeal cancer,” J. Natl. Cancer Inst. 114(4), pp. 609-617, April 2022); variations on SpaTIL for patients who have received immunotherapy (Histo-TIL; see, e.g., Wang, X. et al., “Spatial interplay patterns of cancer nuclei and tumor-infiltrating lymphocytes (TILs) predict clinical benefit for immune checkpoint inhibitors,” Science Advances 8(22), June 2022); and variations on SpaTIL that quantify TIL sub-populations and the interplay between these populations (PhenoTIL; see, e.g., Barrera, C. et al., “Phenotyping tumor infiltrating lymphocytes (PhenoTIL) on H&E tissue images: predicting recurrence in lung cancer,” Proc. SPIE 10956, Medical Imaging 2019: Digital Pathology 1095607, May 2019).

As those of skill in the art will understand, in order to perform a feature extraction, the preprocessor/featurizer elements 22, 24 first perform other tasks. In general, the medical images that are used as input may require quality control or standardization processes before feature extraction can take place. For example, images may need to be upsampled or downsampled to a standard resolution, cropped, or have artifacts removed. As another example, pathology images may be subjected to a quality control process or application prior to use, like the application disclosed in U.S. Pat. No. 10,861,156, the contents of which are incorporated by reference in their entirety.

The medical images may be in essentially any usable format, including Digital Communications in Medicine (DICOM) format, TIFF format, SVS format, JPG format, etc., and may be associated with metadata describing the nature of the study that produced the image, patient information, etc. In non-clinical uses of the foundation model 20, it may be necessary or desirable to anonymize data so as to protect patient identity.

In some cases, the “preprocessing” steps performed on a medical image by preprocessor/featurizer elements 22, 24 may be a function of the file format in which the medical image is stored. For example, in DICOM whole slide imaging of pathology images, lower-resolution versions of a whole slide image are automatically computed and stored in a “pyramid” of image data that facilitates retrieval of image data at arbitrary resolutions.

Beyond the initial preprocessing and quality control of medical images, the preprocessor/featurizer elements 22, 24 will generally also be responsible for segmenting the medical images prior to feature extraction. “Segmentation” is a general term referring to the process of distinguishing the structures in a medical image from one another. This description will generally assume that segmentation is performed automatically, although in some embodiments, the preprocessor/featurizer element 22, 24 could prompt a user to manually segment an image and/or indicate structures of interest using, e.g., a graphical user interface (GUI).

Automatic segmentation methods frequently use deep-learning machine models, such as those based on CNNs, to create a segmentation. Deep-learning machine models specialized for image segmentation, like U-nets, may be used. Other types of segmentation models and approaches that do not rely on artificial neural networks could also be used, including thresholding, active contour, and region-growing approaches.

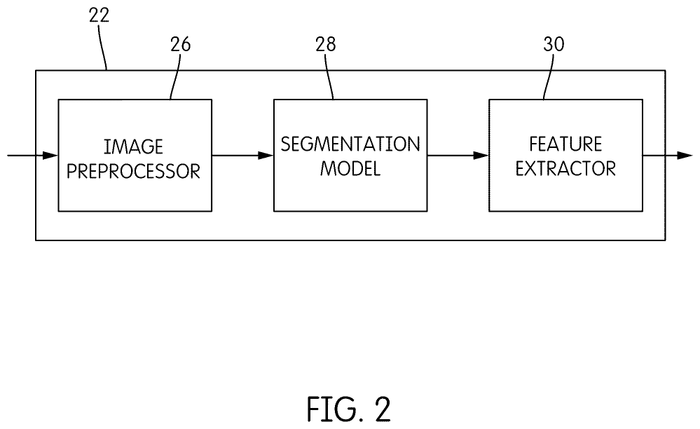

FIG. 2 is a detailed schematic diagram of a preprocessor/featurizer element 22, summarizing its functions. When a medical image is received, an image preprocessor module 26 handles format conversion (if needed), cropping, and quality control. If the medical image is to be processed in portions or tiles, the image preprocessor module 26 may also break the medical image into those portions or tiles. The segmentation model 28 then segments the medical image or its tiles and passes the image and its segmentation to a feature extractor 30 to extract the desired features. Those features may be further processed or encoded, either by the preprocessor/featurizer element 22 or by some other element, before they are passed to the foundation model 20.

When a particular set of radiomic features uses features extracted from a peri-lesional or peri-tumoral region around the lesion or tumor, that peri-lesional or peri-tumoral region may be defined as needed depending on the particular features that are being extracted and other factors. The region may amount to a few millimeters outside the lesion itself, a few centimeters outside the lesion itself, or any other definition that suits the particular type of feature extraction being performed. The contours of the peri-lesional region may also vary. Some feature extraction techniques define the peri-lesional region by dilating the segmented boundaries of the lesion itself, while other techniques may, e.g., simply draw circles or polygons of increasing size centered at the centroid of the lesion. In general, if a particular feature has been shown to be predictive of a particular condition, the feature will generally be defined and extracted in the way known to be most predictive of the particular condition.

Although the use of specific image features may have certain advantages, this is not to say that extracted radiomic or pathomic features must always be used, or that whole medical images or portions of medical images cannot be used without feature extraction. On the contrary, in many cases, the foundation model 20 will be trained to use raw or preprocessed medical images, or at least portions of those images. For example, to make a particular prediction, the foundation model 20 may be passed a set of extracted features and the whole medical image from which those features were extracted (as-is or in patches or tiles), as well as additional genetic, immunohistochemical, or other clinical data for the patient or patients.

For that reason, FIG. 1 shows that there are a number of ingestor/encoder elements 32, 34 designed to accept other forms of medical images and data. Although two ingestor/encoder elements 32, 34 are shown in FIG. 1 for simplicity in illustration, these elements 32, 34 may be numerous in an operational oncological foundation model system 10, each one adapted to accept a different form of medical image or data and to preprocess that medical image or data for use with the foundation model 20. These ingestor/encoder elements 32, 34 might, for example, accept data from a complete blood count (CBC) or a comprehensive metabolic panel (CMP) and extract specific values; parse narrative clinical notes for key words and values and place the extracted text in appropriate form for use with the foundation model 20; parse DNA, RNA, or amino acid sequences, extract specific fragments or portions of the sequences, and place those sequence fragments in appropriate form; parse listings of genetic mutations found in a patient; or consider proteomics or transcriptomics data. Thus, the functions of the ingestor/encoder elements 32, 34 are defined almost entirely by the nature of the data that they are adapted to accept and the nature of the formatting required by the foundation model 20.

Moreover, although much of this description focuses on medical data and images, in some embodiments, the foundation model 20 may be trained to use data originating from more general microbiological and chemical techniques, like chromatographs or data derived from chromatography, and microbiological blots (e.g., Southern blot, Northern blot, etc.) or data derived from those blots. Ingestor/encoder elements 32, 34 may be specifically adapted to work with these types of more generalized data as well.

Depending on the embodiment and the type of data it is designed to accept and to process, an ingestor/encoder 32, 34 may be either a simple algorithmic piece of software, or it may be a machine model (with or without the use of deep learning) that is trained to process that particular form of input data and to produce an output. In some cases, the ingestor/encoder 32, 34 may be a generative machine model that creates a final form of input from a raw or intermediate form of input.

Ultimately, the purpose of input elements 22, 24, 32, 34 is to produce from source data an input suitable for the foundation model 20. To continue the example of transformers given above, these types of models take tokens with positional embedding as input. Thus, the ultimate function of the input elements 22, 24, 32, 34 is to “tokenize” the source data. As one example, a medical image may be divided into tiles or patches, with each of those tiles or patches serving as a token, and the embedding indicating the position of the tile or patch in the greater image. In general, tokenization may be performed in any way known in the art and will vary depending on the nature of the source data. The tokens into which any particular input data is broken or deconstructed may or may not be meaningful to humans.

For example, a sentence can be broken into words, and those words into syllables (or phonemes, for spoken language), and at each level of deconstruction, the fragments of the sentence have human-understandable meaning and function. Similarly, a DNA or RNA sequence can be broken into its individual bases, or into 3-base codons, and an amino acid sequence can be broken into individual amino acids. However, in tokenizing these kinds of data, the preprocessor may break sentences into tokens that are not words, syllables, or phonemes; DNA or RNA into groupings that are not individual bases or codons; and amino acid sequences into groupings that are not individual base pairs. In tokenizing, the focus is on providing the foundation model 20 with the most understandable, actionable input.

In addition to input elements 22, 24, 32, 34 intended to accept data, the system 10 includes an input interface 36 to the foundation model 20. The input interface 36 is designed to gather information from a user (or another source) on what the foundation model 20 is to do with the input data, i.e., what kind of predictions, recommendations, or other outputs are desired. The input interface 36 may, for example, instantiate a graphical or textual user interface on a local device 38 that allows a user to define options and to provide other forms of input. The input interface 36 will typically also produce tokens for input to the foundation model 20, although it may alternatively or additionally influence or determine embeddings for other types of input data.

In a typical embodiment, there may be many different input interfaces, each one distinct from the others, and each operating in parallel to provide input to the foundation model 20 or, at least, to an instance of the foundation model 20. In at least some embodiments, the input interface 36 could be a web server or another such device that can create an interface on a variety of local devices 38 using general purpose software, like a browser. In other embodiments, the local device 38 may have a dedicated application for communicating with the input interface 36. Regardless of the particular type of input interface 36 and the software used on the local device 38, communication between the local device 38 and the input interface 36 may be by standard communication protocols, e.g., HTTP, HTTPS, TCP/IP, UDP, etc. Any communication between the local devices 38 and the input interface 36 may be encrypted and/or otherwise secured.

As was described above, system 10 is designed to integrate data from a plurality of sources and present that data to the foundation model 20, with some form of prediction as output. That data integration may happen overtly, under the control of the user using the input interface 36, or it may happen automatically, based on the input provided to the input interface 36.

In some cases, the input interface 36 may not directly create an interface by itself. For example, the input interface 36 may be an application programming interface (API) that provides routines allowing other software to access the functions of system 10 and the foundation model 20. This type of arrangement would essentially allow the functions of system 10 to be integrated into other medical computing applications, like an electronic health record/electronic medical record (EHR/EMR) system, a radiology information system (RIS), a pharmacy information system, or a computer physician order entry system. With that kind of integration, a medical professional could ask for a prediction from the foundation model 20 while working within a particular patient's chart or with that particular patient's medical images. Whether or not the functions of system 10 are integrated into another type of medical computing application, system 10 preferably has access to an EHR/EMR 40 and/or other types of medical databases, from which it draws information in order to make predictions. The nature of the data repositories that communicate with the foundation model 20 is neither critical nor particularly limited; a data repository may be, e.g., structured or unstructured, and may use any kind of query language. While FIG. 1 shows direct and dedicated connections between the foundation models and certain types of external data repositories, like the EHR/EMR 40, communication may be handled by an API or another type of interface or intermediary module.

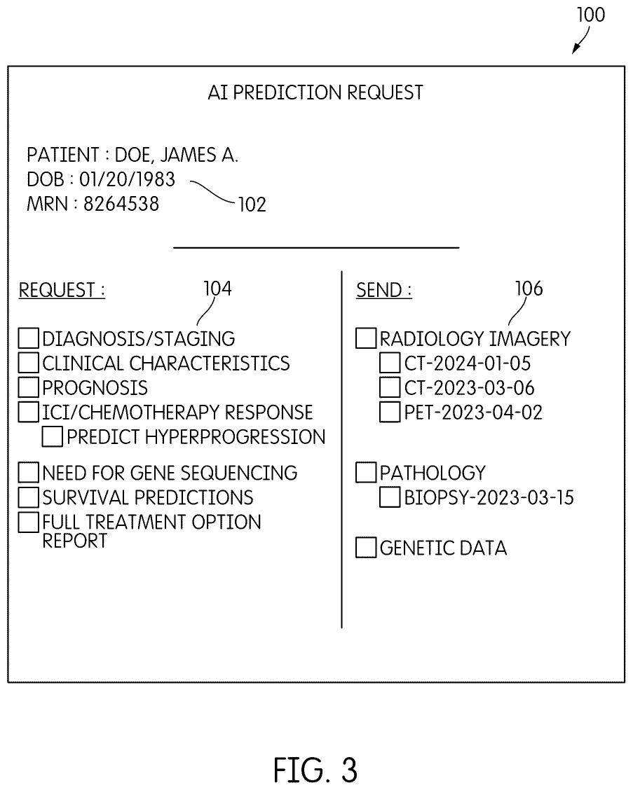

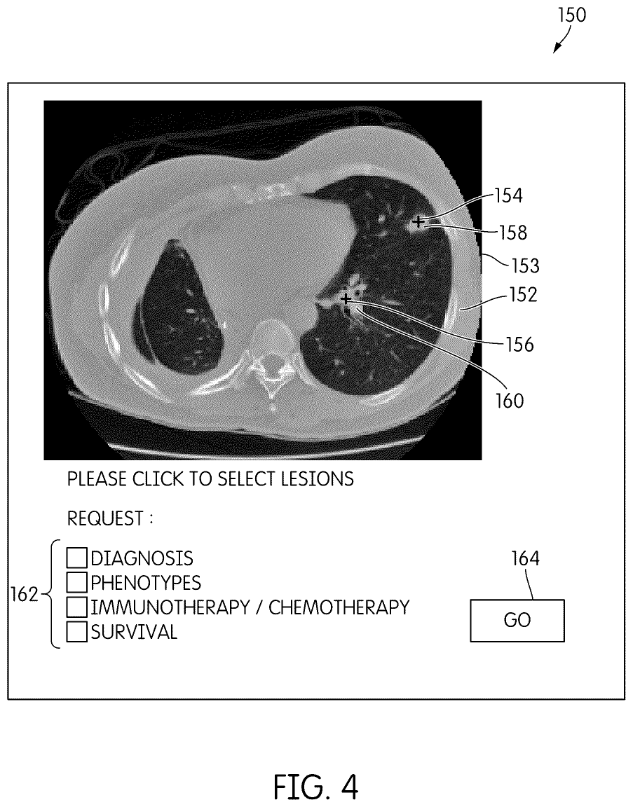

Even with a single foundation model 20, there may be a wide variety of user interfaces 36, i.e., a wide variety of ways in which the foundation model 20 can take input. FIGS. 3 and 4 are schematic illustrations that provide two possible examples.

More specifically, FIG. 3 illustrates a GUI 100. The GUI 100 allows the user to select, using checkboxes, which types of predictions to request from the foundation model 20, and which data to supply to make those predictions. The GUI 100 has a patient identification area 102 that, in this case, displays the patient's name, date of birth, and medical record number. In this example, the request area 104 allows the user to select which predictions to request using checkboxes. Of course, the type of control or selection mechanism is immaterial to the overall function of system 10.

In this example, the user can request a diagnosis (or differential diagnosis) based on a lesion or lesions and staging information; the clinical or phenotypical characteristics of a lesion; the patient's prognosis; a prediction of whether the patient will respond, or respond better to, immunotherapy, such as an immune checkpoint inhibitor (ICI), or to chemotherapy; a prediction of whether a particular treatment is likely to have adverse effects, like hyperprogression; whether the patient is likely to have relevant genetic mutations that should be sequenced; survival predictions, such as overall survival; and full report ranking or weighing various treatment options. To make the requested predictions, the user is permitted to select from the available data in a data selection area 106.

In this example, the data selection area 106 allows the user to send any one of, or a combination of, radiologic, pathologic, and genetic data. In particular, the user is permitted to select which radiology studies to send, and which pathology studies to send. In the illustration of FIG. 3, the user has had two CT scans, spaced apart a little less than year from one another, and a PET scan, as well as a biopsy. There is also genetic data available that can be used for prediction.

In instantiating an interface like the GUI 100, the input interface 36 may draw from available data in an EHR/EMR 40 and dynamically create the GUI 100 to include options only for data that is actually available. The data that is available will vary from patient to patient, depending on what diagnostic procedures, studies, and treatments the patient has had. In this example, all of the available data is routine clinical data gathered in the course of diagnosis, monitoring, and treatment. Even if a particular interface, like the GUI 100, allows a user to select particular data upon which to base a prediction, the input interface 36 or other components of system 10 may cause other data to be passed to the foundation model 20 as well. For example, in all cases, the patient's date of birth, race or ethnicity, sex/gender, and other basic demographic information may be passed to the foundation model 20, with or without that data being explicitly selected.

Several things about the foundation model 20 and the scope of its capabilities can be gleaned from the GUI 100 of FIG. 3. First, some of the information that can be requested from the foundation model 20 using the GUI 100 of FIG. 3 is predictive information that the user would not be able to determine without the assistance of a machine, while other output of the foundation model 20 may be more traditional information that is gathered or used in clinical or research practice. For example, the phenotypical characteristics that the foundation model 20 is trained to produce for a particular lesion would typically align with characteristics that a radiologist or other trained person would appreciate or note for that lesion. Examples include subtlety, structure, calcification, sphericity, margin, lobulation, spiculation, and texture. The output from the foundation model 20 for each of these characteristics may be a score that is indicative of each trait. For example, a high score for sphericity might indicate that a particular lesion is strongly or mostly spherical, while a low score might indicate that a particular lesion is not spherical or is only weakly spherical. A high score for margins might indicate that a particular lesion has clearly defined margins, while a low score might indicate that it does not. However, the “sense” of the score or ranking provided by the foundation model 20 may be normed among the different phenotypical characteristics, such that a high score is always indicative of a more problematic phenotype and a low score is always indicative of a less problematic phenotype, or vice-versa. If the phenotypical characteristic is generally appreciated in clinical practice, then the score will typically follow whatever clinical convention is used for that characteristic. Depending on the nature of the phenotypical characteristic, a score may also be a measurement of that characteristic. (As those of skill in the art will note, if the foundation model 20 this kind of clinical information, the information it provides is generally predictive in nature, i.e., it has an uncertainty associated with it.)

Second, the predictions that can be requested from the foundation model 20 do not necessarily involve the use of generative AI. Rather, many of the predictions involve classifying the available data as belonging to one group or another. The architecture and scope of the foundation model 20 allow the foundation model 20 to provide such predictions with more flexibility than a machine model that does not use deep learning, or even a simple deep learning machine model.

However, as will be described below in more detail, the foundation model 20 may have some generative capabilities in at least some embodiments, or it may be associated with ancillary models that are generative in nature.

For example, if the user requests a prediction as to whether or not the patient has relevant gene mutations and should thus have his or her DNA sequenced to confirm that, the foundation model 20 may be trained to predict whether the user has, e.g., any of 100 relevant genetic mutations. The output of that prediction may be a vector comprising 100 binary values indicating whether or not the patient has each of the 100 relevant genetic mutations. The foundation model 20 may be associated with an ancillary generative machine model that is trained to accept that vector of binary values and to generate a textual or graphical output that is intelligible to medical providers.

In some embodiments, the foundation model 20 may have more extensive generative capabilities. For example, the foundation model 20 may be directed to predict the appearance of chest CT given an existing chest CT or a longitudinal series of chest CTs, or given some other combination of data that may or may not be image-based, like biomarkers or genetic data. The foundation model 20 may be asked to predict pathology or genetic data from radiology medical images. The foundation model 20 may also be used to predict how various characteristics and features of a lesion will evolve over time. For example, given a set of features, such as vessel tortuosity features, the foundation model 20 may be asked to predict how those features will evolve or change over time, given the available data. These sorts of outputs usually involve the use of generative AI.

To at least some extent, the interface created by the input interface 36 and the local devices 38 will reflect the flexibility and range of the foundation model 20, as well as the needs of its users. In the GUI 100, the user is given the opportunity to select the kinds of data that are sent to the foundation model 20. Additionally, some data may be automatically sent to the foundation model 20 regardless of user selections. For example, a certain type of prediction may require certain types of data that are automatically sent when that type of prediction is requested.

The scenario implied by FIG. 3 is, in many cases, what foundation models 20 may excel at—the use of many different types of data to make a prediction. However, there may be situations in which the foundation model 20 is used in more focused ways with fewer types of data. FIG. 4 is an example of this. In FIG. 4, a GUI 150 is integrated with an RIS. In the GUI 150 of FIG. 4, the user views a slice of a thoracic CT scan 152 within a view area 153. In this embodiment, the user is permitted to select points or areas of particular interest, or areas requiring particular attention, using the GUI 150. In this case, the user has clicked on two points 154, 156 within the slice of the CT scan 152. Each of those points 154, 156 coincides with a lesion 158, 160 visible in the slice of the CT scan 152.

Below the view area 153, the user is given a set of controls 162 allowing the selection of desired predictions. In this case, the predictions include: diagnosis, lesion phenotypes, evaluation of predicted response to immunotherapy versus chemotherapy, and survival. In this scenario, the predictions offered may be limited to predictions that can be made with some degree of confidence using only the CT scan or scans available. In other words, while the foundation model 20 used for the predictions may be the same foundation model 20 that was described earlier, this scenario does not assume that other kinds of data beyond the immediate radiology study and medical images are available. Therefore, the predictions offered are limited to things that can be predicted with some degree of confidence solely from radiology data. Once the user has selected all desired predictions, a control 164 sends the data to an appropriate preprocessor/featurizer 22, 24 or ingestor/encoder 32, 34 for preprocessing.

As those of skill in the art will realize, the illustration of FIG. 4 is a simple example of a GUI 150 that is integrated with an RIS. In other embodiments, users may be permitted to select points in multiple slices or portions of a larger radiology study, select regions, outline or trace lesions, measure lesions or their features, etc., all of which could be passed as input to a foundation model 20. In yet other embodiments, if other types of patient data are available, the user may be prompted to include them, or some types of data may be sent to the foundation model 20 automatically.

The points 154, 156 selected by the user in the GUI 150 may be preprocessed and encoded in any number of ways for use by the foundation model 20. In some cases, the points 154, 156 may be encoded as a numerical vector of image coordinates, in either two or three dimensions. In that case, a preprocessor may need to change the frame-of-reference of the coordinates before encoding. In other embodiments, an image distance map or “heat map” could be created, centered on the points 154, 156. In yet other embodiments, a matrix could be constructed that weights each pixel of the input medical image or images according to a distance measure from each point, such as the Euclidean or Chebyshev distance from each point 154, 156 (or one of the points 154, 156) to the pixel in question.

If the foundation model 20 uses specific radiomic features to make predictions from medical images like the medical image 152, then the inputs gathered using the GUI 150 would typically be sent to a preprocessor/featurizer 22, 24 that is adapted to segment the medical image 152 around the points 154, 156 and extract features from the lesion, the peri-lesional area around the lesion, or other regions of the medical image.

FIGS. 3 and 4 are examples of graphical user interfaces 100, 150 used as input interfaces 36 for the foundation model 20. There are many other possible ways in which system 10 can receive input and determine what set of information to send to the foundation model 20. For example, system 10 could receive input in textual form, either as natural language text or as some form of structured text (e.g., XML).

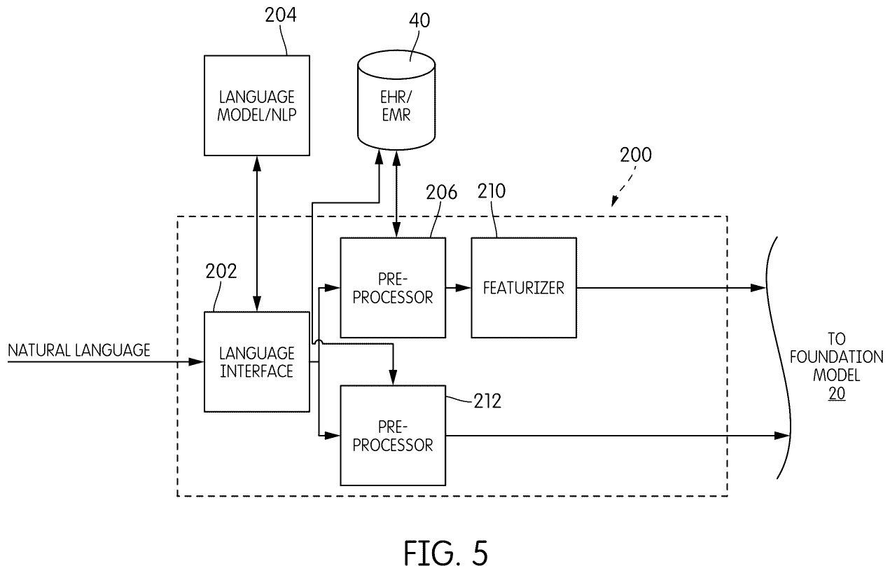

FIG. 5 is a schematic illustration of a combined input interface, ingestor, preprocessor, and featurizer element, generally indicated at 200, according to another embodiment of the invention. More particularly, with reference to FIG. 1, element 200 of FIG. 5 combines elements of an input interface 36, a preprocessor/featurizer 22, and an ingestor/encoder 32. As those of skill in the art will understand, and as FIG. 5 bears out, the functions described with respect to FIG. 1 may be combined in any number of ways as the nature of the input and the nature of the query require.

Element 200 assumes that natural language is used as input, and that at least some of the data is in the form of natural language. For example, instead of selecting or clicking in a graphical user interface, the user may simply type or dictate in natural language. For example, such input may be something like the following:
Patient is a 42-year-old female, medical record number 624338. Mammography and breast tomosynthesis study of February 18 shows heterogeneously dense breasts with an equal-density oval mass measuring 7 millimeters with obscured margins seen in the upper outer quadrant of the left breast, located 6 centimeters from the nipple. This is best visualized on tomosynthesis CC view slice #18 and MLO view slice #24. Please classify the mass and determine whether an ultrasound study or a biopsy is indicated.

The first part of this narrative provides basic information on the patient and on a mass seen in a mammography/breast tomosynthesis study in a format similar to a formal radiology report. The last sentence contains a directive for system 10: “classify the mass and determine whether an ultrasound study or a biopsy is indicated.” Thus, the foundation model 20 is being asked, by this narrative, to determine whether the mass in question is, e.g., a benign breast cyst or a malignant tumor and to determine whether further information is likely to improve the confidence of that prediction.

FIG. 5 illustrates conceptually how such information would be processed. First, if the input is dictated, element 200 may be coupled with a speech-to-text module to translate the audio input to natural language text. FIG. 5 assumes that if that step is necessary, it has already been completed.

In element 200, the natural language input, resulting from typed input or from speech-to-text processing, is received by a language interface 202. That language interface may be, e.g., an API or another such element that connects with and accesses the functions of a language model or a conventional natural language processing (NLP) system 204. Using the language model or NLP system 204, the language interface 202 recognizes the relevant elements of the natural language text. Once that is done, the language interface 202 sends instructions to a preprocessor 206 to retrieve the relevant medical images from an EMR/EHR system 208. Those medical image(s) may be processed by a feature extractor 210 and features extracted before the medical image(s) and extracted features are sent as input to the foundation model 20. (Alternatively, the medical image(s) may be provided to the foundation model 20 by a preprocessing element that does not extract radiomic features, or some combination of features and whole images or image portions may be presented to the foundation model 20, as was described above.)

The query above notes a specific type of radiology study on a specific date in a specific medical record, and data from that specifically-referenced study would generally be retrieved and sent to the foundation model in response to the query, possibly with the particular views noted in the query encoded so as to draw special attention. However, the pre-processor 206 may also retrieve and process other types of studies that may be relevant to the query. This may be done whether or not it is specifically requested. For example, with the above query, the additional data may include longitudinal data, such as any previous mammograms or breast tomosynthesis studies.

Any other non-image medical data relevant to the query may be retrieved and preprocessed by a separate, second preprocessor 212 that is also in communication with the EMR/HER system 40 (or another such data repository) before being sent to the foundation model 20 for use in making a prediction. This may include, e.g., the patient's gender, medical history, recent bloodwork, known genetic or biomarker data, such as BRCA1 and BRCA2 status, etc.

As the above examples attest, the foundation model 20 will often rely on primary sources in making its predictions: original medical images, original bloodwork and biomarker test results, etc. However, it need not be limited to only primary sources. In some cases, secondary sources may also be relied upon to make predictions. These secondary sources could include, for example, formal radiology and pathology reports made by trained personnel who review the primary source material, e.g., the CT scans, MRI scans, whole-slide images of pathology samples, etc. For these purposes, a a medical provider's clinical notes may be either a primary source or a secondary source, depending on whether those notes provide original information on the patient's condition or provide interpretation of, or reflection on, test results, or diagnosis of a particular condition. The foundation model 20 may, in some cases, be trained to weight certain types of data more heavily in making certain types of predictions.

The above examples all detail a single query, the processing of input for that query, and a singular prediction made in response to that query. However, system 10 may be adapted to process multiple queries in bulk, or to process one or more of the same queries in batch form for a plurality of patients. This technique may be used, for example, to divide a large population of patients or potential patients into a group that is likely to respond to a particular drug and a group that is unlikely to respond to a particular drug for purposes of a pharmaceutical drug trial, or for other academic or clinical purposes. If system 10 is to be used to process multiple queries in bulk, or to process one or more of the same queries in batch form, the input interface 36 may be an API, and a batch processing module adapted to make the necessary queries may access system 10 through that API input interface 36. Instructions for such processing may be written and accepted in a scripting language, a data description language, etc.

The foundation model 20 takes whatever data is provided to it, along with instructions for the type and nature of prediction to be made and makes one or more predictions. In most cases, the foundation model 20 can take the input and data provided and make a prediction directly based on the input and data. However, in some cases, the foundation model 20 may make intermediate predictions. For example, if a user asks for a prediction of what a patient's thoracic CT scan will look like in one year based on two previous CT scans of the same area, the foundation model 20 may make a direct generative prediction, or it may engage in a process of successively generating one or more additional predictive scans, e.g., 3 months into the future, 6 months into the future, etc. before generating the final predictive scan to answer the query.

More generally, the foundation model 20 may issue one or more predictions in response to any one query. A query may explicitly call for more than one prediction, a query may implicitly require the foundation model 20 to make a first or intermediate prediction in order to make a prediction that answers the query that was posed, or the foundation model 20 may offer an additional prediction or additional predictions in the name of transparency. For example, the foundation model 20 may automatically predict, without being specifically queried, what form or amount of additional data would allow system 10 to make a particular prediction on a particular topic with greater confidence. Techniques like Bayesian inference could be used to quantify the level of uncertainty for such predictions.

In FIG. 1, the foundation model 20 is shown as outputting predictions via an output interface 42. In a practical implementation of system 10, there may be any number of output interfaces 42, each one adapted or specialized for a particular type of prediction, or to output to a particular type of local device 38. Predictions from the foundation model 20 may also be stored, either in raw form or in processed form, in an EMR/EHR system 40.

As was described above, the immediate output of a prediction may not be readily understandable to, or useable by, an average user. Although much of the output of the foundation model 20 may have a relatable clinical meaning: e.g., a hazard ratio, a risk score, a set of phenotypical characteristics for a lesion, a differential diagnosis or diagnoses, or a prognosis, that does not mean that the form of the output is particularly suited for clinical or research applications. For example, it may be necessary for an output interface 42 to convert the predictive output of the foundation model 20 from one form to another, or to add labels or context, to answer the query.

Simple output-processing tasks may involve, e.g., applying a threshold to a calculated probability score, risk score, or hazard ratio to answer a particular query. For example, if the query is “will this patient respond to nivolumab?” the output from the foundation model 20 is most likely to be some probabilistic score. The output interface 42 may apply a threshold or thresholds to answer that query with “yes,” “likely yes,” “likely no,” “no,” or some other set of defined categories, or to highlight patients who are most likely and least likely to respond in some other way.

The output interface 42 may also add context to a prediction, e.g., by graphing a predictive data point over time to answer a longitudinal query, displaying a predictive data point against a reference range, or highlighting particular areas of an input medical image to display a textual or numerical prediction graphically. In some cases, the output interface 42 may be an ancillary generative model trained to make such graphs and annotations.

To continue in the vein of an example offered above, if the query involves presenting ranked treatment options in response to some set of input medical images and other data, the output of the foundation model 20 may be a vector of numerical values that describe the probability of response to a set of treatment options. A generative output interface 42 may be used to convert those probabilities to a set of textually- or graphically-descriptive options, ranked in order from highest probability to lowest probability, or vice-versa.

The output interface 42 may also be, or be coupled to, a language model or a large language model. This may be particularly helpful if the output of the foundation model 20 is to be a clinical-style report. A clinical report produced by the output interface 42 may follow the general style or conventions of other types of clinical reports, like a radiology report or a pathology report. In addition to the predictive result generated by the foundation model 20, any report may include contextual information, such as an explicit statement of the information that was input and considered in making the prediction, the phenotypical or other characteristics of any lesion or lesions that were considered, the confidence with which the prediction is made, and an indication of any additional data that would allow the foundation model 20 to make the same prediction with greater confidence.

This description refers to “a” foundation model 20 and “the” foundation model 20 in the singular for simplicity in explanation. As those of skill in the art will realize, there may be many instances of a foundation model 20 running simultaneously, or essentially simultaneously, in parallel, each one addressing a different query or a group of related queries, and each one connected to and/or in communication with different input elements 22, 24, 32, 34, 36, different input and output interfaces 36, 38, and ultimately, different local devices 38.

### Training of Oncological Foundation Models

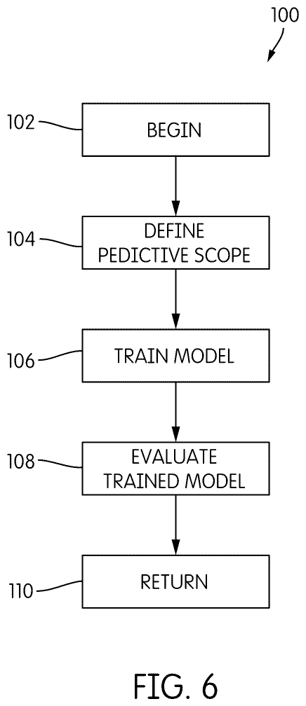

FIG. 6 is a schematic flow diagram of a method, generally indicated at 100, for training an oncological foundation model 20. Method 100 begins at 102 and continues with 104.

The first task of any training method, indicated at task 104, is to define the scope of prediction, i.e., the nature and scope of the queries that the foundation model 20 is expected to answer. For example, if a foundation model is expected to answer a query based on thoracic CT images, it will be trained with thoracic CT images. If a foundation model is expected to answer a query based on a combination of thoracic CT images and whole-slide images of lung tissue, it will be trained on both of those things. As those of skill in the art will appreciate, a foundation model 20 should generally be trained on data that is as close as possible to the actual data that will be provided to the foundation model 20 in answering “live” queries.

Hand-in-hand with defining the scope of prediction, task 104 may involve defining the architecture of the foundation model 20 and any associated elements, including any preprocessor/featurizer elements 22, 24, any ingestor/encoder elements 32, 34, and any interfaces 36, 42. As was noted above, while much of this description assumes that the foundation model 20 has a transformer-based architecture, other architectures may be used. Additionally, as was also described above, the foundation model 20 may use and be associated with other, more focused models that are used to make specific kinds of predictions, or with language models or other generative models that are used, e.g., as output interfaces 42.

Once the scope of the foundation model 20 is established in task 104, method 100 continues with task 106, and training begins. Because the foundation model will typically be adapted to answer queries concerning a range of different types of cancers using a wide range of different kinds of data, it will usually be trained with medical images (e.g., at least CT, MRI, PET, and X-ray) of various portions of the body, showing healthy patients as well as patients with various forms and stages of disease. The majority of that disease will most likely be cancers of various sorts, stages, and treatments, but a foundation model should also be trained with images showing various presentations and stages of every likely differential diagnosis, as well as images showing complications of various treatments.

Similarly, the foundation model will typically be trained with pathology images, such as whole-slide images, showing various types of specimens taken from patients with various types and stages of cancers. As was noted earlier, many of those specimens would be routine clinical specimens stained with H&E stain, but other types of micrographs and documentary photographs may be used, and a foundation model may be trained with micrographs of specially-prepared and stained tissue.

For example, a foundation model 20 may be trained to make predictions concerning one or more of 10 types of cancer that result in solid tumors: breast, lung and bronchus, colon and rectal, urinary bladder, thyroid, kidney and renal, pelvis, uterine corpus, oral cavity and pharynx, and ovarian. Given that, a dataset comprising the complete records of about 15,000 patients may be acquired and used in training. This dataset would include at least radiology images, pathology images, immunohistochemical and biomarker data, and genetic data.

While foundation models are typically trained using self-supervised learning, foundation models 20 according to this description may be trained, at least in part, using supervised learning techniques. In general, supervised learning uses labeled data to train a machine model. That labeled data may associate a particular medical image with a diagnosis, stage, complication, or other form of outcome; contain annotations indicating the positions of lesions in a medical image; associate a particular medical image with the presence or absence of certain genetic mutations; etc. The labeled data may, in some cases, be used in a particular order during training. For example, a foundation model 20 could be trained on a larger corpus of unlabeled data using self-supervised techniques, and then fine-tuned with labeled data.

The use of supervised learning with labeled data may reduce the overall data needs for training, allowing the use of a smaller training corpus of data. In one embodiment, about 10-25% of the training may be supervised learning, with the training data labeled, e.g., by outcome. The more outcome labels are used, the more those predictions can inform not just the predictive components of the foundation model 20, but also the embeddings/representations of each modality. Without the use of labeled data in training, there is a greater reliance on the foundation model 20 to learn useful representations purely based on relationships in and between modalities.

The training data set may use traditional augmentation techniques to expose the foundation model 20 to as many variations and perturbations of data as possible. In creating a functional foundation model 20, it may be helpful to train with one type or modality of data at a time and to verify the ability of the foundation model 20 to answer queries correctly using that type or modality of data before training on another type or modality of data.

Any known training techniques may be used. For example, as was noted above, it may be helpful if the final model is as small as possible in order to facilitate transparency. However, in some embodiments, a large foundation model (i.e., with more parameters than the final foundation model is intended to have) could be constructed, and the technique of knowledge distillation could be used in training, in which a smaller model would be trained to predict the output of the larger model. When trained, that smaller model would be deployed as the foundation model 20.

In some cases, the training data and methods may be different from what the foundation model 20 is designed to accept in practice. For example, the description above focuses, in part, on the use of features extracted from medical images as input to the foundation model 20, in addition, or as an alternative, to having the foundation model 20 process whole medical images. As was described above, not all embodiments of a foundation model 20 need use image features. However, even if a foundation model 20 is not trained to use image features in making predictions, image features may be extracted and used in training. For instance, image features could be used to teach an image-based foundation model better representations, and to reduce training needs.

Once a foundation model 20 is trained, it is evaluated, as shown in task 108 of method 100. It is not necessary for all of the training to be completed in task 106 before evaluation begins in task 108. Rather, as was described above, foundation models 20 will typically be trained in stages, with a model 20 trained, e.g., on one modality or type of data before progressing to the next modality or type of data. For that reason, foundation models 20 may be evaluated in stages, with a foundation model 20 trained in one modality or type of data and then evaluated with respect to that modality or type of data.

In simple cases, evaluation may involve using a new corpus of patient data, not previously used for training, with known outcomes relative to the queries that are to be answered. The foundation model 20 would then be evaluated on its ability to correctly predict the known outcomes based on the patient data.

As the foundation model 20 is asked to make complex predictions, there may not be a known outcome in the corpus of data used for evaluation. Thus, in complex cases, similarity scores could be calculated that describe the similarity between the patient for whom the foundation model 20 is being asked to make a prediction and other patients for whom outcomes are known. Ultimately, essentially any evaluation methods may be used.

Method 100 concludes at 110.

This description assumes that the foundation model 20 is a singular model. In practice, several models may be trained simultaneously, and the model that shows the best performance in task 108 could be used as the foundation model 20.

### Causal Prediction and Training

As those of skill in the art will note, some of the types of predictions described here are causal predictions, e.g., predictions that quantify changes in outcomes due to various treatments. In essence, causal predictions allow one to address counterfactual situations in ways that conventional machine learning prediction may not, e.g., to estimate the effects of a particular treatment. Whereas more conventional machine learning prediction might, for example, predict which patients are at high risk for a particular type of cancer, or which patients might benefit most from immunotherapy, causal machine learning may be used for more complex, comparative predictions. As one example, casual machine learning and causal prediction may be used to select the most effective treatment for the patient while at the same time minimizing adverse effects due to treatment. In a specific case, e.g., causal machine learning may be used to predict whether chemotherapy alone, immunotherapy alone, or a combination of immunotherapy and chemotherapy is likely to be most effective with minimal side effects.

The training of a foundation model 20 for causal prediction generally follows the basic tasks set forth in method 100, with some additional considerations. For example, in task 104, defining the predictive scope of the foundation model 20 would generally also involve formulating the causal structure of the problem or query to be addressed, selecting the causal quantity or quantities of interest (e.g., the individualized effect of a particular treatment), and defining the information necessary to make a causal prediction. For example, in one case, it may be desirable to predict the size of a solid tumor as an outcome if a patient is given a particular type of chemotherapy. The covariates in this case would be the patient's previous medical history. Any previous treatments that were given could also be introduced to the model as covariates.

Formulating the causal structure of the problem also necessarily involves assumptions as to causality, e.g., that certain covariates, like age, gender, and medical history, and particular treatments do influence the outcome that one wishes to predict. In task 104, the plausibility of any such assumptions should be evaluated.

In causal prediction, the treatment in question may be either binary or continuous. For example, a binary treatment may be a case in which a treatment is either provided or not provided. A continuous treatment is one for which there are a continuous range of treatment possibilities, e.g., a selectable radiation dose, or a selectable chemotherapy dose. For a continuous treatment, the potential outcomes are multidimensional.

During training, the foundation model 20 would be trained with enough data from different patients with different outcomes to be able to predict counterfactuals. As was described above, during evaluation in task 108, similarity scores could be calculated that map the covariates in any prediction to patients with known outcomes.

### Longitudinal Prediction

As was described above, a longitudinal prediction is one that concerns the evolution or progression of a patient or a group of patients over time. Longitudinal predictions may be causal in some cases, and they may involve the use of generative AI in some cases. For example, a foundation model 20 may be asked to predict the appearance of thoracic CT scans over time if chemotherapy is given versus if immunotherapy is given. That prediction involves both causal prediction-used to evaluate the effect of one treatment versus another- and generative AI.

Generally speaking, longitudinal data may be used much like any other data, and longitudinal predictions may be made much like any other predictions. The main difference is that as new data is input to the foundation model 20, it is encoded as input to the model in a way that distinguishes it with respect to time from other data.

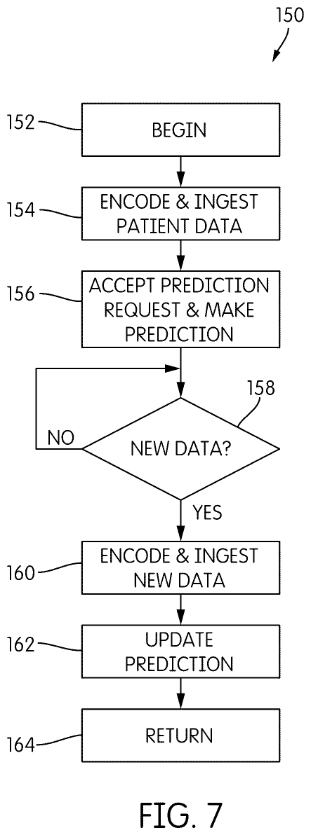

FIG. 7 is a brief schematic flow diagram illustrating a method, generally indicated at 150, for making and updating longitudinal predictions. Method 150 begins at task 152 and continues with task 154. In task 154, the foundation model 20 encodes and ingests patient data as well as the prediction request in task 156. As was described briefly above, when longitudinal data is ingested, it is encoded as to time, so that, e.g., medical images from one study are differentiated from medical images from another study 3 or 6 months later. If the prediction to be made relies on radiomic or pathomic features extracted from a medical image by a preprocessor/featurizer 22, 24, any extracted features would be similarly encoded to distinguish them from features or images from earlier or later medical studies.

In task 156, the foundation model 20 makes a prediction. That prediction may include a confidence interval, a suggestion as to other data that might improve the prediction quality, a graph, etc., all of which could be produced by an output interface 42. In many cases, once a longitudinal prediction is made, a method like method 150 may end; that is, there may be no need for further prediction.

However, with longitudinal predictions, when new data is received, a prior prediction may be updated to reflect new longitudinal data. Method 150 as illustrated in FIG. 7 assumes that as new data is received, prior predictions are updated. In practice, if a prediction is to be updated with new data, it may be done manually or automatically. That is, in some cases, a user may push a button, click a control, or otherwise make a manual request that a prior prediction be updated. In other cases, the system 10 may automatically preprocess, featurize, and ingest new data from an EHR as it is generated from various kinds of medical studies and update any predictions that have been made, or at least, some subset of predictions for which updates have been requested.

Task 158 is a decision task. If new data has been generated and received, (task 158: YES), method 150 proceeds with task 160 and that new data is preprocessed (if needed) and encoded appropriately to indicate its time-relation with other data. In task 162, an updated prediction is made. In some cases, this may entail presenting the foundation model 20 with all of the old data and the new data and asking for a new prediction, and in other cases, the foundation model 20 may already have access to the old data and may only be presented with the new data.

All references cited herein are hereby incorporated by reference in their entireties.

While the invention has been described with respect to certain embodiments, the description is intended to be exemplary, rather than limiting. Modifications and changes may be made within the scope of the invention, which is defined by the appended claims.

What is claimed is:

1. A system comprising:
an oncological foundation model, implemented on one or more processors, the oncological foundation model trained to make predictions concerning two or more different types of cancers using two or more different types or modalities of input data.

2. The system of claim 1, wherein the two or more different types or modalities of data are selected from the group consisting of radiology images, pathology images, genetic data, proteomic data, and transcriptomic data.

3. The system of claim 2, wherein the radiology images are images resulting from CT scans, MRI scans, PET scans, or X-rays.

4. The system of claim 2, wherein the pathology images are whole-slide images of tissue samples.

5. The system of claim 1, further comprising:
at least one preprocessor/featurizer element coupled to the oncological foundation model, the preprocessor/featurizer element adapted to:
(a) extract features from a medical image; and
(b) prepare the extracted features for input to the oncological foundation model.

6. The system of claim 5, wherein the medical image is a radiology image and the features are radiomic features.

7. The system of claim 5, wherein the medical image is a pathology image and the features are pathomic features.

8. The system of claim 1, wherein the two or more different types of cancers comprise solid tumors of the breast, lung, bronchus, colon, rectum, urinary bladder, thyroid, kidney, pelvis, uterine corpus, oral cavity, and ovary.

9. The system of claim 1, wherein the predictions comprise causal predictions comparing effects of two or more different treatments.

10. The system of claim 1, wherein at least some of the predictions further comprise a confidence estimate.

11. A method comprising:
receiving, at an oncological foundation model, a first set of data comprising one or both of a medical image or a set of features derived from the medical image;
receiving, at the oncological foundation model, a second set of data distinct from the first set of data; and
providing one or more predictions based on the first set of data and the second set of data.

12. The method of claim 11, wherein the medical image of the first set of data comprises a radiology or pathology medical image.

13. The method of claim 12, wherein the second set of data is selected from the group consisting of genetic data, immunohistochemistry data, biomarker data, medical history data, and patient demographic data.

14. The method of claim 13, wherein the second set of data comprises medical history data, and the medical history data comprises one or more past medical treatments.

15. The method of claim 11, further comprising providing a confidence estimate related to the one or more predictions.

16. A method of training an oncological foundation model, comprising:
training at least one deep learning machine model using two or more different types or modalities of data relating to two or more different types of cancers using both self-supervised and supervised learning;
wherein the two or more different types or modalities of data comprise two or more of radiology data, pathology data, genetic data, immunohistochemistry data, biomarker data, medical history data, or patient demographic data.

17. The method of claim 16, wherein the deep learning machine model comprises a transformer-based deep learning machine model.

18. The method of claim 16, wherein the two or more different types or modalities of data further comprises longitudinal data.

19. The method of claim 16, wherein said training comprises:
constructing a large deep learning machine model using the two or more different types or modalities of data relating to the two or more different types of cancers;
constructing a small deep learning machine model, the small deep learning machine model having fewer parameters than the large deep learning model, such that the small deep learning model is trained to emulate the large deep learning machine model; and
deploying the small deep learning model as the oncological foundation model.

20. The method of claim 16, wherein the two or more different types or modalities of data include one or more sets of features derived from medical images, the one or more sets of features having an established association with a particular biological phenomenon or effect.
